(19) United States
(12) Patent Application Publication
(10) Pub. No.: US 2017/0080035 A1
(43) Pub. Date: Mar. 23, 2017

(54) PHARMACEUTICAL COMPOSITION FOR TREATING ROTAVIRUS AND/OR RESPIRATORY SYNCYTIAL VIRUS INFECTION AND METHOD FOR PREPARING THE SAME

(71) Applicants: Far East Bio-Tec. Co., Ltd., Taipei (TW); National Taiwan University, Taipei (TW)

(72) Inventors: Chuang Chun CHIUH, Taipei (TW); Chun Nan LEE, Taipei (TW); Chun Ting CHENG, Taipei (TW)

(21) Appl. No.: 15/365,071

(22) Filed: Nov. 30, 2016

Related U.S. Application Data
(62) Division of application No. 12/461,306, filed on Aug. 7, 2009.

(30) Foreign Application Priority Data
Aug. 11, 2008 (TW) 97130505

(51) Int. Cl. A61K 35/748

(52) U.S. Cl. CPC A61K 35/748

(57) ABSTRACT

The present invention provides a pharmaceutical composition for treating rotavirus and/or respiratory syncytial virus infection and a method for preparing the same; wherein said pharmaceutical composition comprises a therapeutically effective amount of Spirulina extract and a pharmaceutically acceptable carrier. The Spirulina extract used in the present invention is extracted at a low temperature by the following steps: (a) adding an organic Spirulina powder into a hypotonic buffer solution and mixed well to obtain a mixture; (b) freezing the mixture from step (a) overnight at a temperature lower than 0° C.; (c) thawing the mixture from step (b); (d) separating and purifying the mixture from step (c) by a separator, and collecting blue fractions; and (e) spray drying the blue fractions from step (d).

### BACKGROUND OF THE INVENTION

Field of the Invention

The present invention relates to a pharmaceutical composition, particularly a pharmaceutical composition for treating rotavirus and/or respiratory syncytial virus infection. The present invention also relates to a method for preparing a pharmaceutical composition for treating rotavirus and/or respiratory syncytial virus infection.

Description of the Related Art

Acute infectious diarrhea is a leading cause of disease or death in many countries of the world. Diarrhea problem is quite serious in developing countries. As in Asia, Africa, and Latin America, there are three to four billion diarrhea cases every year, in which five to ten million patients die (see Walsh, J. A. et al. N. Eng. J. Med., 301: 967-974, 1979). Now it has recognized that rotavirus is one of the major causes of serious diarrhea in infants and babies (see Estes, M. K. Rotaviruses and Their Replication in Fields Virology, Third Edition, edited by Fields et al., Raven Publisher, Philadephia, 1996). According to the statistical data, rotavirus causes more than one million deaths every year.

Additionally, respiratory syncytial virus (RSV) is the main cause of viral pneumonia and bronchitis in infants and babies, in which infected infants aged from six weeks to six months usually have quite serious symptoms. Therefore, research and development of a new drug for preventing or treating rotavirus and/or respiratory syncytial virus is one of the important researches in the world.

There are two commercial oral vaccines against rotavirus in the market, and they are Rotarix® (produced by GSK) and RotaTeq® (produced by MSD). The research reports of these two vaccines both have proved that their preventing effects reach 95%, but this protection can be maintain for only months, and the recipients also take the risk of intussusception. The main treatment for rotavirus now is a supporting therapy. There is no approved inhibiting drug against rotavirus.

No exactly effective vaccine against respiratory syncytial virus has been developed. Now the only way to prevent RSV infection is utilizing passive immunity of RSV neutralizing antibody, such as RSV-IVIG (brand name: RespiGam®) and palivizumab (brand name: Synagis®), but the effect is quite limited. As for treatment, the only approved anti-RSV drug is a nucleotide preparation, Ribavirin (brand name: virazole), and its effect still needs to be improved. Therefore, developing a novel anti-RSV drug is necessary.

### SUMMARY OF THE INVENTION

Accordingly, the present invention provides a novel pharmaceutical composition for treating rotavirus and/or respiratory syncytial virus infection, which is prepared by obtaining a Spirulina extract by low temperature extraction, and mixing said extract with a pharmaceutically acceptable carrier.

One object of the present invention is to provide a pharmaceutical composition comprising a Spirulina extract, which has an ability to inhibit rotavirus and/or respiratory syncytial virus infection and/or replication.

Another object of the present invention is to provide a method for preparing the above-mentioned pharmaceutical composition.

To achieve these objects, the present invention provides a pharmaceutical composition for treating rotavirus and/or respiratory syncytial virus infection, comprising a therapeutically effective amount of Spirulina extract and a pharmaceutically acceptable carrier.

In a preferred embodiment, the pharmaceutical composition further comprises a pharmaceutically acceptable adjuvant, excipient or additive.

In a preferred embodiment, the pharmaceutical composition is in a form of powder, particle, liquid, gel, or paste.

In a preferred embodiment, the pharmaceutical composition is provided in a dosage form of food, beverage, medicine, reagent, or nutritional supplement.

In a preferred embodiment, the pharmaceutical composition is administered to a subject via oral administration, injection, inhalation, subcutaneous implantation, or skin patch.

In a preferred embodiment, the pharmaceutical composition is suitable for preventing and/or treating rotavirus and/or respiratory syncytial virus infection.

In a preferred embodiment, the pharmaceutical composition is suitable for inhibiting virus infection and/or replication.

The present invention also provides a method for preparing a pharmaceutical composition for treating rotavirus and/or respiratory syncytial virus infection, wherein said pharmaceutical composition comprises a therapeutically effective amount of Spirulina extract and a pharmaceutically acceptable carrier, and said Spirulina extract is extracted at −25° C. to 18° C. by the following steps: (a) adding an organic Spirulina powder into a hypotonic buffer solution and mixed well to obtain a mixture; (b) freezing the mixture from step (a) overnight at a temperature lower than 0° C.; (c) thawing the mixture from step (b);

(d) separating and purifying the mixture from step (c) by a separator, and collecting blue fractions; and (e) spray drying the blue fractions from step (d). Preferably, the mixture from step (a) is frozen overnight at a temperature of −25° C. to −10° C. Also preferably, the mixture from step (b) is thawed at 4° C. to 18° C.

In the present invention, a Spirulina extract is obtained by a low temperature extraction step, and the pharmaceutical composition comprising the extract is administered to a subject infected by rotavirus and/or respiratory syncytial virus to inhibit rotavirus and/or respiratory syncytial virus infection and/or replication effectively. In addition, the bioactivity of the active ingredient comprised in the Spirulina extract can be maintained because said Spirulina extract is extracted at a low temperature.

### BRIEF DESCRIPTION OF THE DRAWINGS

FIG. 1 represents MTT assay results of HEp-2 and MA104 cells.

FIG. 2A represents the rotavirus cytopathic effects inhibited by Spirulina extract at different concentrations, in which a is cell control group, b to e are virus groups comprising Spirulina extract at concentrations of 3.125, 0.781, 0.195 and 0.049 mg/mL, and F is virus control group.

FIG. 2B represents the respiratory syncytial virus cytopathic effects inhibited by Spirulina extract at different concentrations, in which a is cell control group, b to e are virus groups comprising Spirulina extract at concentrations of 3.125, 0.781, 0.195 and 0.049 mg/mL, and F is virus control group.

FIG. 3 represents respiratory syncytial virus inhibition of Spirulina extract determined by MTT assay.

FIG. 4 represents rotavirus inhibition of Spirulina extract determined by fluorescent focus reduction assay.

### DETAILED DESCRIPTION OF THE PREFERRED EMBODIMENTS

Spirulina extract is used as the effective ingredient of the pharmaceutical composition of the present invention. Since Spirulina extract has an ability to inhibit rotavirus and/or respiratory syncytial virus infection and/or replication, it is applied to provide a novel drug for treating rotavirus and/or respiratory syncytial virus infection.

The present invention provides a pharmaceutical composition for treating rotavirus and/or respiratory syncytial virus, comprising a therapeutically effective amount of Spirulina extract and a pharmaceutically acceptable carrier. Said pharmaceutical composition may further optionally comprise a pharmaceutically acceptable adjuvant, excipient or additive.

The “carrier” used herein may comprise an inert component. The inert component does not substantially react with other ingredients comprised in the pharmaceutical composition of the present invention. The standard techniques for preparing a pharmaceutical dosage form, as described in Remington's Pharmaceutical Sciences, Mack Publishing Company, Easton, Pa., can be used. Appropriate pharmaceutical carrier comprises, but not limited to, e.g. sterile water, normal saline, phosphate buffer saline, Hanks' solution, lactose-Ringer solution, or other conventional carriers used in pharmaceutical technology.

The “excipient” used herein may has a variety of functions and purposes. For example, a disintegrating agent can be added during the preparation of an oral dosage form, such as tablet, to disintegrate the tablet into small particles for absorption in gastrointestinal tract; and a coloring agent can be added to improve the appearance. Other parental preparation, such as injection, suspension, ointment, suppository, spray and the like, can be combined with a suitable excipient for its specific use. Appropriate excipient should comprises, but not limited to, e.g., lactose, mannitol, glucan, glucose, glutamic acid, gelatin, sorbitol, fucose, sucrose, xylitol, starch, microcrystalline cellulose, methyl cellulose, Acacia gum, or combinations thereof. The use of excipient is a general knowledge of this technical art.

The “effective amount” used herein refers to a compound dosage resulting in advantageous benefits when the compound is provided to a subject, or a compound dosage brining the expected activity in vivo or in vitro. In contrast to the untreated patients suffering from, e.g. influenza, the advantageous clinical benefits of treated influenza patients comprise alleviating symptoms, ameliorating discomfort, reducing progression, accelerating healing, and the like. The precise dosage providing to a subject depends on the disease, the progression or symptom of the disease and the physical conditions of the subject such as the health, age, gender, weight, and drug tolerance during administration. The dosage is also associated with the progression, severity and classification of the disease. Appropriate dosage can be decided by those skilled in the art according to the above-mentioned or other factors.

Yet the present invention provides a method for preparing a pharmaceutical composition for treating rotavirus and/or respiratory syncytial virus, wherein said pharmaceutical composition comprises a therapeutically effective amount of Spirulina extract and a pharmaceutically acceptable carrier, and said Spirulina extract is extracted at −25° C. to 18° C. by the following steps: (a) adding an organic Spirulina powder into a hypotonic buffer solution and mixed well to obtain a mixture; (b) freezing the mixture from step (a) overnight at a temperature lower than 0° C.; (c) thawing the mixture from step (b); (d) separating and purifying the mixture from step (c) by a separator, and collecting blue fractions; and (e) spray drying the blue fractions from step (d).

The “hypotonic buffer solution” used herein refers to a human edible solution having a lower osmotic pressure than organic Spirulina. For example, the hypotonic buffer solution comprises, but not limited to, pure water, 0.1% sodium chloride solution, 0.3% sodium chloride solution, or 0.5% glucose solution.

The examples of this invention are provided hereinafter, however, these examples are not used for limit the present invention. Any amendments and modifications can be made by those skilled in the art without departing the spirit and scope of the present invention. The scope of the present invention is defined by the appended claims.

### EXAMPLES

### Experimental Materials

Cell: MA104 (a fetal rhesus monkey kidney cell line) and HEp-2 (a human laryngeal cancer cell line)

Virus strain: human rotavirus Wa and G9 (VR010591)

respiratory syncytial virus, a clinical strain from National Taiwan

University Hospital, Taiwan.

Composition to be tested: a Spirulina extract solution comprising Spirulina extract and sterile PBSA, i.e. the pharmaceutical composition of the present invention.

### Experiment Methods

### A. Cell Culture

Cell lines MA104 and HEp-2, which can be used for in vitro continuous culture, were frozen and stored in liquid nitrogen. These cells were rapidly thawed, centrifuged to remove cryopreservation solution, and then added growth medium comprising fetal calf serum, and the resulting mixture was moved to a cell culture flask and incubated under 5% CO2 at 37° C. in an incubator. These cells were subcultured once every 3-4 days. When mono-layered cells grew to full confluence in the flask, the culture medium was removed, and the cells were washed by phosphate buffer solution (PBS) twice. Trypsin was then added to react for 5 to 10 minutes. When the cells were detached, growth medium was added and well-mixed with the cells. After that, cells were counted and diluted to an appropriate concentration for consequential virus culture or other tests.

### B. Virus Culture

Human rotavirus (HRV) was inoculated into MA104 cells, and respiratory syncytial virus was inoculated into HEp-2 cells. Rotavirus was activated by trypsin at 37° C. for 30 minutes before inoculation. After inoculation, the cells were incubated at 37° C. for virus absorption. One hour later, the medium was changed to fresh maintenance medium, and the viruses were incubated under 5% CO2 at 37° C. in an incubator, in which rotavirus was incubated on a roller. HEp-2 cells were incubated until more than 75% of cells represented syncytial cytopathic effects (CPE), and MA104 cells were incubated until more than 75% of cells were broken and detached. After those, the viruses were collected from these cells by freeze-thaw cycles, and the virus solutions were centrifuged at 5,000 rpm for 10 minutes at 4° C. to remove cell debris. The virus culture supernatant, in which cells had been removed, was so-called virus solution in the following examples, and it was aliquoted into 3 mL small glass tube and stored at −80° C.

### C. Preparation of Spirulina Extract Solution

The Spirulina extract powder was diluted by sterile PBSA into a concentration of 25 mg/mL, and then it was directly stored at −20° C. without filtration. In the following examples, this storage solution was diluted into Spirulina extract solutions at appropriate concentrations.

### D. Primary Assessment of Virus Inhibition Effect of Spirulina Extract In Vitro

MA104 or HEp-2 cells at an appropriate concentration were cultured in a glass tube. On the second day, Spirulina extract serially diluted by maintenance medium was mixed with the virus solution of rotavirus or respiratory syncytial virus at a certain concentration in a ratio of 1:1. The mixture was reacted at 37° C. for 30 minutes, and then 100 μL of the mixture was used to infect the cells grown on the bottom of the glass tubes at full confluence. The viruses were infected at 37° C. for 1 hour, and then 1 mL maintenance medium comprising the Spirulina extract at the same concentration was added. The cytopathic effect of the cells was observed every day.

### E. Cytotoxicity of Spirulina Extract Determined by MTT Assay

MA104 or HEp-2 cells at an appropriate concentration were cultured in 96-well plates. On the second day, 200 μL of Spirulina extract serially diluted by maintenance medium was added in the 96-well plates on which the cells were at full confluence. After 3 days culture, 5 mg/mL of MTT was added. The cells were maintained at 37° C. for 5 hours, and then DMSO as a lysis buffer was added and the cells were further maintained at 37° C. for 10 minutes. After that, the OD570 absorbance of each well was determined.

### F. Virus Inhibition Effect of Spirulina Extract Determined by MTT Assay

### Experiment before Virus Absorption

MA104 or HEp-2 cells at an appropriate concentration were cultured in 96-well plates. On the second day, Spirulina extract serially diluted by maintenance medium was mixed with the virus solution of rotavirus or respiratory syncytial virus at a certain concentration in a ratio of 1:1. The mixture was reacted at 37° C. for 30 minutes, and then 100 μL of the mixture was used to inoculate the cells grown in the 96-well plates at full confluence. The virus absorption was processed at 37° C. for 1 hour, then the virus solution was suctioned, and 200 μL maintenance medium comprising no Spirulina extract was added. After 3 days culture, MTT was added. The cells were maintained at 37° C. for 5 hours, and then a lysis buffer (DMSO) was added and the cells were further maintained at 37° C. for 10 minutes. After that, the OD570 absorbance of each well was determined.

### Experiment after Virus Absorption

MA104 or HEp-2 cells at an appropriate concentration were cultured in 96-well plates overnight. On the second day, each well was inoculated with 100 μL of virus solution and maintained at 37° C. for 1 hour for virus absorption. Next, the virus solution was suctioned, and 200 μL Spirulina extract serially diluted by maintenance medium was added. After 3 days culture, MTT was added. The cells were maintained at 37° C. for 5 hours, and then a lysis buffer (DMSO) was added and the cells were further maintained at 37° C. for 10 minutes. After that, the OD570 absorbance of each well was determined.

### G. Rotavirus Inhibition Effect of Spirulina Extract Determined by Focus Reduction Assay

### Experiment before Virus Absorption

MA104 cells at an appropriate concentration were cultured in 96-well plates overnight. On the second day, Spirulina extract serially diluted by maintenance medium was mixed with the rotavirus solution at a certain concentration in a ratio of 1:1. The mixture was reacted at 37° C. for 30 minutes, and then 100 μL of the mixture was used to inoculate the cells grown in the 96-well plates at full confluence. The virus absorption was processed at 37° C. for 1 hour, then the virus solution was suctioned, and 200 μL maintenance medium comprising no Spirulina extract was added. After 18-24 hours culture, the supernatant was suctioned and ice-cold methanol was added to fix the cells for 20 minutes. The 96-well plates were placed face-down to dry. Subsequently, a 1:40 dilution of rabbit anti-HRV serum was added, and the cells were incubated at 37° C. for 2 hours. The cells were washed by PBSA for three times. Then a 1:150 dilution of FITC-conjugated goat anti-rabbit IgG (Zymed) was added, and the cells were incubated at 37° C. for 1 hour in the dark. After that, the cells were washed by ddH2O for three times. The plates were placed face-down to dry, and the fluorescent cells were counted by fluorescence microscopy.

### Experiment after Virus Absorption

MA104 or HEp-2 cells at an appropriate concentration were cultured in 96-well plates overnight. On the second day, 100 μL of virus solution was used to inoculate the cells in each well. The virus absorption was processed at 37° C. for 1 hour, then the virus solution was suctioned, and 200 μL Spirulina extract serially diluted by maintenance medium was added. After 18-24 hours culture, the supernatant was suctioned and ice-cold methanol was added to fix the cells for 20 minutes. The 96-well plates were placed face-down to dry. Subsequently, a 1:40 dilution of rabbit anti-HRV serum was added, and the cells were incubated at 37° C. for 2 hours. The cells were washed by PBSA for three times. Then a 1:150 dilution of FITC-conjugated goat anti-rabbit IgG was added, and the cells were incubated at 37° C. for 1 hour in the dark. After that, the cells were washed by ddH2O for three times. The plates were placed face-down to dry, and the fluorescent cells were counted by fluorescence microscopy.

### Experimental Results

### 1. Cytotoxicity of Spirulina Extract

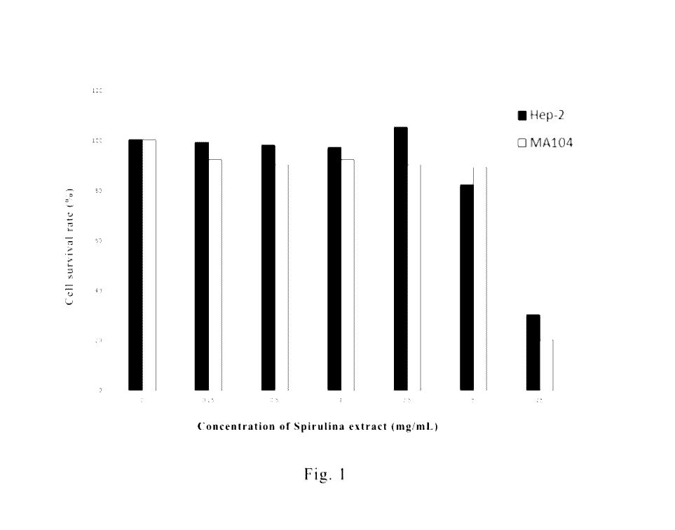

Dilutions of Spirulina extract at different concentrations were added to pre-cultured cells, and the cell activity was determined by MTT assay. FIG. 1 represented the MTT assay results of HEp-2 and MA104 cells. Compared with the control cells, the undiluted Spirulina extract (i.e. the maximum concentration, 25 mg/mL) had a significant cytotoxicity on either HEp-2 or MA104 cells. In other words, the cell survival rate was lower than 50%. Spirulina extract at the concentration lower than 5 mg/mL had no significant cytotoxicity. As shown in FIG. 1, when Spirulina extract at the concentration of 25 mg/mL was used, the cell survival rate was lower than 50%. However, this was because Spirulina extract at the maximum concentration (25 mg/mL) was diluted by PBSA, in which no culture medium was comprised, so the cells were died because of growth difficulty. From the experiment results, we knew that the Spirulina extract itself had no significant toxicity to the cells.

### 2. Rotavirus and Respiratory Syncytial Virus Cytopathic Effects Reduced by Spirulina Extract

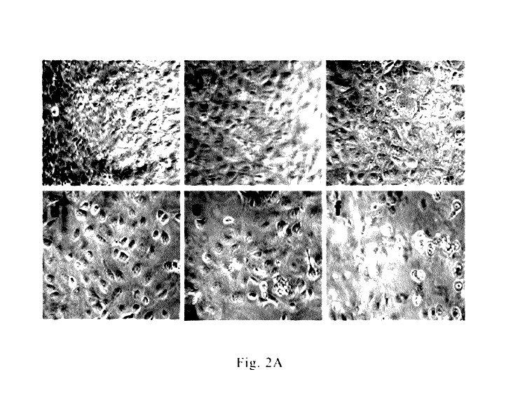

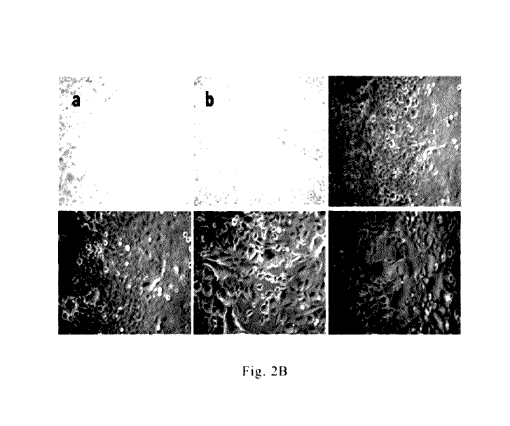

Rotavirus or respiratory syncytial virus was cultured in vitro, and Spirulina extract at different concentrations were added or not added during incubation. Cytopathic effects of the cells were observed everyday, and recorded by microscopy at the seventh day after rotavirus infection and at the second day after respiratory syncytial virus infection. The results were shown in FIGS. 2A and 2B, respectively. FIG. 2A represented the inhibition of rotavirus cytopathic effects by Spirulina extract at different concentrations, and FIG. 2B represented the inhibition of respiratory syncytial virus cytopathic effects by Spirulina extract at different concentrations. The results of FIGS. 2A and 2B showed that Spirulina extract at specific concentrations could effectively inhibit virus cytopathic effects, and the inhibition level was positively related to the concentration of Spirulinaextract.

### 3. Respiratory Syncytial Virus Inhibition of Spirulina Extract Determined by MTT Assay

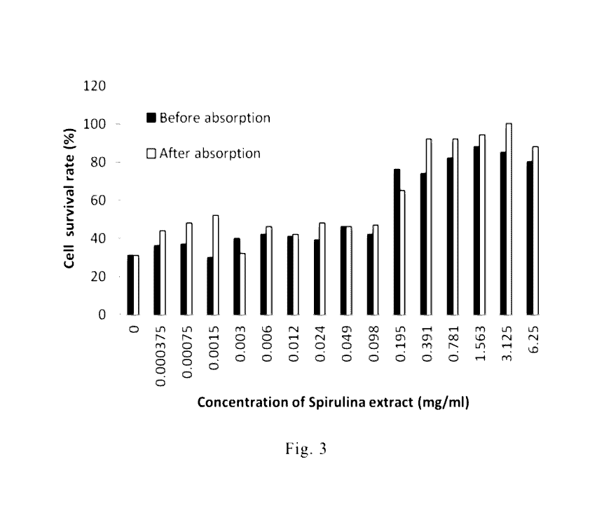

100 TCID50 of respiratory syncytial virus was used to infect HEp-2 cells grown in a 96-well plate at full confluence. 2× serially diluted Spirulina extracts were mixed with virus solution before virus infection, or added in maintenance medium after virus infection, and then MTT assay was processed separately. The results were shown in FIG. 3. FIG. 3 represented that the observed cell survival rate was about 33% when the concentration of Spirulina extract was 0. In other words, the cells were infected by virus while no Spirulina extract was exited, so the cells were killed directly. In addition, the inhibition effects of respiratory syncytial virus by Spirulina extract before and after virus absorption were similar. The lowest concentration of Spirulina extract to inhibit 50% infection of virus (IC50) was about 0.195 mg/mL, and when Spirulina extract at a higher concentration was used, the cell activity could be maintained at 80% or more.

### 4. Rotavirus Inhibition of Spirulina Extract Determined by Fluorescent Focus Reduction Assay

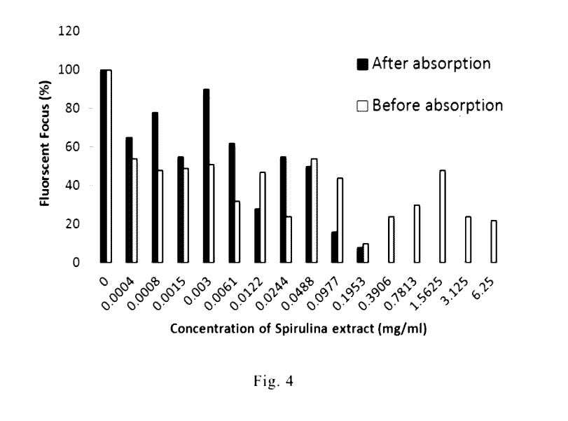

100 pfu of rotavirus is used to infect MA104 cells grown in 96-well plates at full confluence. 2× serially diluted Spirulina extracts were mixed with virus solution before virus infection, or added in maintenance medium after virus infection, and then fluorescent focus reduction assay was processed separately. The results were shown in FIG. 4. In FIG. 4, the data derived when the concentration of Spirulina extract was 0 was defined as virus control. It should be appreciated from FIG. 4 that administration after virus infection could effectively inhibit the formation of fluorescent focus, and there was an approximately positive relation between this inhibition and the concentration of Spirulina extract. In other words, the lowest concentration of Spirulina extract to inhibit 50% infection of virus (IC50) was about 0.049 mg/mL. And, administration before virus infection also inhibited rotavirus infection.

From above, the present invention provides a pharmaceutical composition comprising Spirulina extract, which has an ability to inhibit rotavirus and/or respiratory syncytial virus infection and/or replication, and administering Spirulina extract either before or after virus infection can achieve the effect of inhibiting virus infection and/or replication. Therefore, the pharmaceutical composition of the present invention is suitable for preventing and/or treating rotavirus and/or respiratory syncytial virus infection and/or replication.

### Other Embodiments

All characteristics disclosed herein can be combined in any combination. And the characteristics disclosed herein can be replaced by any characteristics to be used for the same, equal, or similar purpose. Thus, unless otherwise specified, the characteristics disclosed herein are examples of a series of equal or similar characteristics.

In addition, based on the disclosure herein, appropriate changes and modifications may be made by those skilled in the art according to different uses and situations without departing from the spirit and scope of the invention. Therefore, other embodiments are included in the claims of the present invention.

What is claimed is:

1. A method for treating virus infections, comprising administrating a therapeutically effective amount of a Spirulina extract and a pharmaceutically acceptable carrier to a subject.

2. The method of claim 1, wherein the virus includes respiratory syncytial virus or rotavirus.

3. The method of claim 1, wherein the Spirulina extract is extracted at −25° C. to 18° C. by the following steps (a) adding an organic Spirulina powder into a hypotonic buffer solution and mixing well to obtain a mixture;
(b) freezing the mixture from step (a) overnight at a temperature lower than 0° C.
(c) thawing the mixture from step (b);
(d) separating and purifying the mixture from step (c) by a separator, and collecting blue fractions; and
(e) spray drying the blue fractions from step (d).

4. The method of claim 1, wherein the treating includes prevention and curing.

5. The method of claim 4, wherein the prevention and curing includes inhibit virus infection.

6. The method of claim 1, wherein the administrating via oral, injection, inhalation, subcutaneous implantation, or skin patch.

7. The method of claim 1, wherein when the virus is the respiratory syncytial virus, the therapeutically effective amount of Spirulina extract is 0.003 mg/mL to 25 mg/mL.

8. The method of claim 1, wherein when the virus is the rotavirus, the therapeutically effective amount of Spirulina extract is 0.0004 mg/mL to 25 mg/mL.
